(19) United States
(12) Patent Application Publication
(10) Pub. No.: US 2011/0135574 A1
(43) Pub. Date: Jun. 9, 2011

(54) METHODS OF TREATING KIDNEY DISEASE

(76) Inventor: ANNA GREKA, Milton, MA (US)

(21) Appl. No.: 12/947,574

(22) Filed: Nov. 16, 2010

Related U.S. Application Data
(60) Provisional application No. 61/261,603, filed on Nov. 16, 2009.
(60) Provisional application No. 61/261,966, filed on Nov. 17, 2009.

(51) Int. Cl. A61K 49/00; A61K 39/395; A61K 39/00; A61K 31/713; A61P 13/12; G01N 33/53

(52) U.S. Cl. 424/9.2; 424/130.1; 424/184.1; 514/44 A; 435/7.21

(57) ABSTRACT

It has been discovered that TRPC5 activity abolishes actin stress fibers and diminishes focal adhesion formation, rendering a motile, migratory podocyte phenotype. This invention relates generally to methods of reducing expression or activity of TRPC5 to treat, or reduce risk of developing, kidney disease, e.g., proteinuria, in a subject.

### CROSS REFERENCE TO RELATED APPLICATIONS

This application claims priority to U.S. Application Nos. 61/261,603, filed on Nov. 16, 2009, and 61/261,966, filed on Nov. 17, 2009, each of which is incorporated herein by reference in its entirety.

### TECHNICAL FIELD

This invention relates to methods of treating kidney disease, e.g., proteinuria, by inhibiting TRPC5.

### BACKGROUND

Proteinuria is a condition in which an excessive amount of protein in the blood leaks into the urine. Proteinuria can progress from a loss of 30 mg of protein in the urine over a 24 hour period (called microalbuminuria) to >300 mg/day (called macroalbuminuria), before reaching levels of 3.5 grams of protein or more over a 24 hour period, or 25 times the normal amount. Proteinuria occurs when there is a malfunction in the kidney's glomeruli, causing fluid to accumulate in the body (edema). Prolonged protein leakage has been shown to result in kidney failure. Nephrotic Syndrome (NS) disease accounts for approximately 12% of prevalent end stage renal disease cases at an annual cost in the United States of more than $3 billion. Approximately 5 out of every 100,000 children are diagnosed with NS every year and 15 out of every 100,000 children are living with it today. For patients who respond positively to treatment, the relapse frequency is extremely high. 90% of children with Nephrotic Syndrome will respond to treatment, however, an estimated 75% will relapse. Therefore, more effective methods of treating, or reducing risk of developing, kidney disease, e.g., proteinuria, are desirable.

### SUMMARY

This invention is based, at least in part, on the discovery that Transient Receptor Potential Cation Channel, subfamily C, member 5 (TRPC5) activity abolishes actin stress fibers and diminishes focal adhesion formation, rendering a motile, migratory podocyte phenotype.

In one aspect, the invention features methods of treating, or reducing risk of developing, kidney disease (e.g., proteinuria, microalbuminuria, macroalbuminuria) in a subject by administering a therapeutically effective amount of a TRPC5 inhibitor to the subject. The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

In some embodiments, the methods include administering an anti-TRPC5 antibody or antigen-binding fragment thereof.

In some embodiments, the methods include administering an inhibitory nucleic acid (e.g., a small interfering RNA molecule or antisense nucleic acid) that specifically targets TRPC5.

In some embodiments, the methods include administering 2-aminoethoxydiphenylborane and 1-oleoyl-2-acetyl-sn-glycerol.

In some embodiments, the methods include detecting TRPC5 levels in a sample comprising podocytes, comparing TRPC5 levels in the sample to a reference level of TRPC5, and administering the TRPC5 inhibitor if the levels of TRPC5 in the sample are elevated as compared to the reference.

In one aspect, the invention features methods of identifying candidate compounds that reduce or inhibit proteinuria. The methods include contacting a sample (e.g., a living cell) comprising a TRPC5 polypeptide with a test compound and determining a level of calcium (Ca²⁺) transport in the sample in the presence of the test compound. If the test compound decreases the level of calcium transport, relative to a level of calcium transport in the absence of the test compound, then the test compound is a candidate compound for the inhibition of proteinuria.

In some embodiments, the method further comprises administering the candidate compound to a mammal and evaluating an effect of the candidate compound on calcium transport, wherein a candidate compound that reduces or inhibits calcium transport is a candidate therapeutic agent for the treatment of proteinuria.

The invention provides several advantages. The prophylactic and therapeutic methods described herein using a TRPC5 inhibitor are effective in treating kidney disease, e.g., proteinuria, and have minimal, if any, side effects. Further, methods described herein are effective to identify candidate compounds that reduce or inhibit proteinuria.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### DESCRIPTION OF DRAWINGS

FIG. 1. AngII-evoked TRPC-like currents in podocytes. (A) Fluo 3 Ca²⁺ imaging in AT1R podocytes revealed reproducible Ca²⁺ transients in response to AngII. The Ca²⁺ ionophore Ionomycin was added to the bath at the end of the experiment to calibrate the response. (B) Normalized fluorescence amplitude of the calcium response to AngII application (F). Application of the AT1R antagonist Losartan (Los, 200 nM) reduced the amplitude of the AngII-induced Ca²⁺ transient (control, n=15; Losartan, n=14). (C) Whole cell recording of AngII-evoked TRPC-like current in AT1R podocytes. (D) Normalized current (n=5) showed a significant increase in both inward and outward currents after 500 nM AngII application to the bath. (E) TRPC5 and TRPC6 contribute to the AngII-evoked Ca²⁺ transients in podocytes. Representative traces for TRPC5 (n=12) and TRPC6 (n=15) shRNAs. TRPC1 (n=16) or TRPC7 (n=14) shRNAs have no significant effect on AngII-evoked Ca²⁺ transient. (F) Summary data of normalized fluorescence (F). Gene silencing of TRPC5 and TRPC6 resulted in a 60% and 80% decrease in the amplitude of the Ca²⁺ transient, respectively, as compared to scrambled shRNA controls (n=13). For all assays, One-way ANOVA, Fisher's LSD, P<0.001).

FIG. 2. TRPC5-like and TRPC6-like single channel recordings in podocytes. (A) Representative single channel current traces in outside-out patches with voltage steps from −100 mV to +100 mV in AT1R podocytes (V_{hold}−60 mV). (B-D) At Vₛₜₑₚ+100 mV, application of AngII (500 nM) to the bath evoked significant single channel opening (B). This single channel activity was suppressed by 100 μM La³⁺, (C) in a reversible manner (D). The middle panel shows frequency histograms of single channel open probability Pₒ (pin size 0.1 sec) under each condition. The lower panel is the single channel amplitude frequency distribution histogram, from which single channel conductance was calculated from the fitted curve. The single channel conductances were [1] 39 pS, [2] 68 pS, [3] 80 pS and [4] 33 pS.

FIG. 3. AngII-induced TRPC5 and TRPC6 activation has antagonistic effects on the actin cytoskeleton. (A) Serum starved wild-type (WT) and AT1R podocytes displayed prominent stress fibers at baseline (a, d). AngII (500 nM) induced reorganization of the actin cytoskeleton into actin arcs and loss of stress fibers (b, e). Exposure to serum resulted in prominent actin arc formation (c, f; scale bar, 20 μm). (B) Losartan (200 nM) rescued the phenotype by restoring stress fibers (d) and focal adhesions (e) throughout the cytoplasm (scale bar, 20 μm). (C) TRPC6 silenced podocytes (C6 shRNA) displayed loss of stress fibers after AngII treatment. Paxillin staining was also reduced compared to scrambled shRNA controls (Scr shRNA). TRPC5 depleted AT1R podocytes (C5 shRNA) had increased stress fibers and paxillin positive structures throughout the cytoplasm (c, f, i; scale bar, 20 μm). (D) Quantification of stress fibers in AT1R cells (see text for details). (E) There was no significant difference in focal adhesions between TRPC5 shRNA cells and scrambled shRNA controls, but TRPC6 cells displayed significantly reduced focal contacts (see text for details). (F-I) RT-PCR analysis in podocytes revealed that gene silencing of TRPC5 did not result in reciprocal upregulation of TRPC6 (F), or vice versa (G, quantified in H, I).

FIG. 4. Functional coupling of TRPC5 to Rac1 and TRPC6 to RhoA. (A) GTPase activity assay in HEK cells. AngII treatment increased Rac1-GTP levels in cells that co-express TRPC5-GFP and AT1R (Lane 2), but reduced Rac1-GTP in cells that co-express TRPC6-GFP and AT1R (Lane 3). AngII treatment increased RhoA-GTP levels in TRPC6/AT1R cells (Lane 3), but reduced RhoA-GTP in TRPC5/AT1R cells (Lane 2). Data are normalized to total Rac1 and RhoA cell lysate levels (Lanes 1-5). GTP□S-loaded cells served as a positive control (Lane 5). No Rac1-GTP was observed in HEK293T cells that do not express AT1R (Lane 4). TRPC5-GFP and TRPC6-GFP expression was confirmed by blotting with anti-GFP antibody (Lanes 2 and 3). (B, C) Quantification of activity assays (One way ANOVA, Newman-Keuls Multiple Comparison test, P<0.001, n=6). (D) Relative to control podocytes (a), DN RhoAN19 abolished stress fibers (b). DN RacN17 rescued stress fiber and actin arc formation (c) (scale bar, 20 μm). AngII-treated TRPC5 depleted cells displayed prominent stress fibers (d). DN RhoN19 abrogated C5 shRNA mediated stress fibers (e), as did inhibition of RhoA activity by ROCKi (f). AngII-treated TRPC6 silenced podocytes displayed profound loss of stress fibers (g). DN RacN17 promoted stress fiber and actin arc formation in C6 shRNA cells (h). Restoration of TRPC6 expression in the background of C6 shRNA (C6 sRNA+WT C6) also restored actin arc and stress fiber formation (i) (scale bar, 20 μm). (E) Quantification of stress fibers in podocytes (see text for details).

FIG. 5. Role of TRPCs in cell migration. (A) Wound assays at t=36 hours. In control Scr shRNA podocytes, DN RhoN19 induced modest cell migration into the wound (b). TRPC5 depleted cells attenuated AT1R podocyte migration into the wound (c), which was partially reversed by the introduction of DN RhoN19 (d). DN RacN17 abrogated migration into the wound (e). TRPC6 depleted cells showed increased individual cell migration into the wound, compared to controls and TRPC5 depleted cells (f). DN RacN17 on a C6 shRNA background impaired the ability of cells to migrate into the wound (g), mirrored in cells expressing WT TRPC6 on a background of C6 shRNA (h) (scale bar 1 mm). (B) Quantification of migrating podocytes (see text for details). (C) Role of TRPCs in Swiss 3T3 fibroblast migration. Swiss 3T3 cells showed significant migration into the wound 36 hours after introduction of serum (a). Expression of DN RhoN19 induced similar levels of cell motility (b), but introduction of DN RacN17 abrogated migration into the wound (c). As in podocytes, stationary TRPC5 depleted cells did not migrate (d), but motility was restored by expression of DN RhoN19 (e). In contrast, TRPC6 depleted cells showed increased cell migration (f) relative to baseline (a), which was abrogated by DN RacN17 (g) (scale bar, 1 mm). (D) Quantification of migrating fibroblasts (see text for details).

FIG. 6. A model for the role of TRPC5 and TRPC6 in the regulation of actin dynamics and cell motility. (A) Under baseline, physiologic conditions, stimulation of G-protein coupled receptor activates both TRPC6 and TRPC5 channels, allowing the influx of Ca²⁺ into the cell. Ca²⁺, an inherently localized, transient signal, promotes the GTP-bound state of the small GTPases RhoA and Rac1. Activated Rac1 and RhoA are known to be mutually inhibitory. Our data suggest that TRPC5 activation augments Rac1-GTP, which inhibits RhoA and mediates stress fiber disassembly, thus promoting cell motility. On the contrary, TRPC6 increases RhoA-GTP, which inhibits Rac1 activity and promotes stress fiber formation, thus mediating a contractile cell phenotype. After treatment with AngII, TRPC5 mediated Rac1-GTP and TRPC6 mediated RhoA-GTP are in balance, resulting in the control cell phenotype characterized by both actin arcs and stress fibers. (B) Gene silencing of TRPC5 tips the balance in favor of a TRPC6/RhoA dominant phenotype, which renders the cell more contractile. (C) In contrast, gene silencing of TRPC6 tips the balance in favor of a TRPC5/Rac1 phenotype, which renders the cell more motile.

FIG. 7. (A) RT-PCR analysis for TRPC channels in wild type and AT1R podocytes. TRPC1, TRPC5, TRPC6 and TRPC7 mRNA are detected. AT1R podocytes additionally express TRPC4. (B) RT-PCR validation of shRNA-mediated gene silencing of TRPCs in HEK cells. Data indicate three of five most efficient (40-70% knockdown) shRNA sequences (a-e) specifically targeted to each TRPC. In subsequent experiments, the three constructs were introduced simultaneously into podocytes to achieve optimal knockdown efficiency at 80-90% (see also FIG. 4F-I). (C) RT-PCR in Swiss 3T3 fibroblast cells detected TRPC4, TRPC5 and TRPC6 transcripts.

FIG. 8. (A) Wild-type TRPC6 silenced podocytes (C6 shRNA) displayed loss of stress fibers after AngII treatment. Paxillin staining was also reduced compared to scrambled shRNA controls (Scr shRNA). TRPC5 depleted AT1R podocytes (C5 shRNA) had increased stress fibers and paxillin positive structures throughout the cytoplasm (scale bar, 20 μm). (B) Quantification of stress fibers in wild-type cells (see text for details). (C) Quantification of stress fibers in wild-type cells (see text for details). (D) TRPC1 or TRPC7 shRNA did not result in significant morphology changes for the actin cytoskeleton in AT1R podocytes treated with AngII as compared to scrambled shRNA controls.

FIGS. 9A and B. Expression of WT C6 on a C6 shRNA background (n=14) restored the amplitude of the calcium transient in AT1R podocytes as compared to C6 shRNA controls (n=19) (P<0.001).

FIG. 10. (A) Serum induced migration of podocyte monolayers at t=0, 36 and 100 hours, across an artificial 2 mm wound at 100× magnification. In control experiments, podocytes migrate slowly across the wound (a-c). At t=36 hours, after addition of serum, podocytes migrate as a sheet of cells (b). The wound closes 100 hours after addition of serum (c) (scale bar, 1 mm). (B) 400× magnification images of the wound edge at t=36 hours. Cells in conditions permissive of migration display characteristic membrane ruffling and filopodia, as in Scr shRNA controls (a), DN RhoN19 (b), C5 shRNA+DN RacN17, and C6 shRNA (f). On the contrary, cells unable to migrate display a lack of membrane ruffling or filopodia, as in C5 shRNA (c), Scr shRNA+DN RacN17 (e), C6 shRNA+DN RacN17 (g), and C6 shRNA+WT C6 (h) (scale bar, 20 μm). The asterisk denotes the location of the wound.

### DETAILED DESCRIPTION

The non-selective Ca²⁺-permeable Transient Receptor Potential (TRP) channels act as sensors that transduce extracellular cues to the intracellular environment in diverse cellular processes, including actin remodeling and cell migration (Greka et al., Nat Neurosci 6, 837-845, 2003; Ramsey et al., Annu Rev Physiol 68, 619-647, 2006; Montell, Pflugers Arch 451, 19-28, 2005; Clapham, Nature 426, 517-524, 2003). Dynamic rearrangement of the actin cytoskeleton relies on spatiotemporally regulated Ca²⁺ influx (Zheng and Poo, Annu Rev Cell Dev Biol 23, 375-404, 2007); Brandman and Meyer, Science 322, 390-395, 2008); Collins and Meyer, Dev Cell 16, 160-161, 2009) and the small GTPases RhoA and Rac1 serve as key modulators of these changes (Etienne-Manneville and Hall, Nature 420, 629-635, 2002); Raftopoulou and Hall, Dev Biol 265, 23-32, 2004). RhoA induces stress fiber and focal adhesion formation, while Rac1 mediates lamellipodia formation (Etienne-Manneville and Hall, Nature 420, 629-635, 2002). The Transient Receptor Potential Cation Channel, subfamily C, member 5 (TRPC5) acts in concert with TRPC6 to regulate Ca²⁺ influx, actin remodeling, and cell motility in kidney podocytes and fibroblasts. TRPC5-mediated Ca²⁺ influx increases Rac1 activity, whereas TRPC6-mediated Ca²⁺ influx promotes RhoA activity. Gene silencing of TRPC6 channels abolishes stress fibers and diminishes focal contacts, rendering a motile, migratory cell phenotype. In contrast, gene silencing of TRPC5 channels rescues stress fiber formation, rendering a contractile cell phenotype. The results described herein unveil a conserved signaling mechanism whereby TRPC5 and TRPC6 channels control a tightly regulated balance of cytoskeletal dynamics through differential coupling to Rac1 and RhoA.

Ca²⁺-dependent remodeling of the actin cytoskeleton is a dynamic process that drives cell migration (Wei et al., Nature 457, 901-905, 2009). RhoA and Rac1 act as switches responsible for cytoskeletal rearrangements in migrating cells (Etienne-Manneville and Hall, Nature 420, 629-635, 2002); Raftopoulou and Hall, Dev Biol 265, 23-32, 2004). Activation of Rac1 mediates a motile cell phenotype, whereas RhoA activity promotes a contractile phenotype (Etienne-Manneville and Hall, Nature 420, 629-635, 2002). Ca²⁺ plays a central role in small GTPase regulation (Aspenstrom et al., Biochem J 377, 327-337, 2004). Spatially and temporally restricted flickers of Ca²⁺ are enriched near the leading edge of migrating cells (Wei et al., Nature 457, 901-905, 2009). Ca²⁺ microdomains have thus joined local bursts in Rac1 activity (Gardiner et al., Curr Biol 12, 2029-2034, 2002; Machacek et al., Nature 461, 99-103, 2009) as critical events at the leading edge. To date, the sources of Ca²⁺ influx responsible for GTPase regulation remain largely elusive. TRP (Transient Receptor Potential) channels generate time and space-limited Ca²⁺ signals linked to cell migration in fibroblasts and neuronal growth cones⁰. Specifically, TRPC5 channels are known regulators of neuronal growth cone guidance¹ and their activity in neurons is dependent on PI3K and Rac1 activity (Bezzerides et al., Nat Cell Biol 6, 709-720, 2004).

Podocytes are neuronal-like cells that originate from the metanephric mesenchyme of the kidney glomerulus and are essential to the formation of the kidney filtration apparatus (Somlo and Mundel, Nat Genet. 24, 333-335, 2000; Fukasawa et al., J Am Soc Nephrol 20, 1491-1503, 2009). Podocytes possess an exquisitely refined repertoire of cytoskeletal adaptations to environmental cues (Somlo and Mundel, Nat Genet 24, 333-335, 2000; Garg et al., Mol Cell Biol 27, 8698-8712, 2007; Verma et al., J Clin Invest 116, 1346-1359, 2006; Verma et al., J Biol Chem 278, 20716-20723, 2003; Barletta et al., J Biol Chem 278, 19266-19271, 2003; Holzman et al., Kidney Int 56, 1481-1491, 1999; Ahola et al., Am J Pathol 155, 907-913, 1999; Tryggvason and Wartiovaara, N Engl J Med 354, 1387-1401, 2006; Schnabel and Farquhar, J Cell Biol 111, 1255-1263, 1990; Kurihara et al., Proc Natl Acad Sci USA 89, 7075-7079, 1992). Early events of podocyte injury are characterized by dysregulation of the actin cytoskeleton (Faul et al., Trends Cell Biol 17, 428-437, 2007; Takeda et al., J Clin Invest 108, 289-301, 2001; Asanuma et al., Nat Cell Biol 8, 485-491, 2006) and Ca²⁺ homeostasis (Hunt et al., J Am Soc Nephrol 16, 1593-1602, 2005; Faul et al., Nat Med 14, 931-938, 2008). These changes are associated with the onset of proteinuria, the loss of albumin into the urinary space, and ultimately kidney failure (Tryggvason and Wartiovaara, N Engl J Med 354, 1387-1401, 2006). The vasoactive hormone Angiotensin II induces Ca²⁺ influx in podocytes, and prolonged treatment results in loss of stress fibers (Hsu et al., J Mol Med 86, 1379-1394, 2008). While there is a recognized link between Ca²⁺ influx and cytoskeletal reorganization, the mechanisms by which the podocyte senses and transduces extracellular cues that modulate cell shape and motility remain elusive. TRP Canonical 6 (TRPC6) channel mutations have been linked to podocyte injury (Winn et al., Science 308, 1801-1804, 2005; Reiser et al., Nat Genet 37, 739-744, 2005; Moller et al., J Am Soc Nephrol 18, 29-36, 2007; Hsu et al., Biochim Biophys Acta 1772, 928-936, 2007), but little is known about the specific pathways that regulate this process. Moreover, TRPC6 shares close homology with six other members of the TRPC channel family (Ramsey et al., Annu Rev Physiol 68, 619-647, 2006; Clapham, Nature 426, 517-524, 2003). TRPC5 channels antagonize TRPC6 channel activity to control a tightly regulated balance of cytoskeletal dynamics through differential coupling to distinct small GTPases.

### Proteinuria

Proteinuria is a pathological condition wherein protein is present in the urine. Albuminuria is a type of proteinuria. Microalbuminuria occurs when the kidney leaks small amounts of albumin into the urine. In a properly functioning body, albumin is not normally present in urine because it is retained in the bloodstream by the kidneys. Microalbuminuria is diagnosed either from a 24 hour urine collection (20 to 200 μg/min) or, more commonly, from elevated concentrations (30 to 300 mg/L) on at least two occasions. Microalbuminuria can be a forerunner of diabetic nephropathy. An albumin level above these values is called macroalbuminuria. Subjects with certain conditions, e.g., diabetic nephropathy, can progress from microalbuminuria to macroalbuminuria and reach a nephrotic range (>3.5 g/24 hours) as kidney disease reaches advanced stages.

### Causes of Proteinuria

Proteinuria can be associated with a number of conditions, including focal segmental glomerulosclerosis, hypertensive disorder of pregnancy, IgA nephropathy, IgM nephropathy, diabetic nephropathy, lupus nephritis, Goodpasture's syndrome, membranoproliferative glomerulonephritis, poststreptococcal glomerulnephritis, progressive (crescentic) glomerulonephritis, and membranous glomerulonephritis.

A. Focal Segmental Glomerulosclerosis (FSGS)

Focal Segmental Glomerulosclerosis (FSGS) is a disease that attacks the kidney's filtering system (glomeruli) causing serious scarring. FSGS is one of the many causes of a disease known as Nephrotic Syndrome, which occurs when protein in the blood leaks into the urine (proteinuria).

Very few treatments are available for patients with FSGS. Many patients are treated with steroid regimens, most of which have very harsh side effects. Some patients have shown to respond positively to immunosuppressive drugs as well as blood pressure drugs which have shown to lower the level of protein in the urine. To date, there is no commonly accepted effective treatment or cure and there are no FDA approved drugs to treat FSGS. Therefore, more effective methods to reduce or inhibit proteinuria are desirable.

B. Hypertensive Disorder of Pregnancy

Pre-eclampsia is a medical condition where hypertension arises in pregnancy (pregnancy-induced hypertension) in association with significant amounts of protein in the urine. Because pre-eclampsia refers to a set of symptoms rather than any causative factor, it is established that there are many different causes for the syndrome. It also appears likely that there is a substance or substances from the placenta that may cause endothelial dysfunction in the maternal blood vessels of susceptible women. While blood pressure elevation is the most visible sign of the disease, it involves generalized damage to the maternal endothelium and kidneys and liver, with the release of vasopressive factors only secondary to the original damage.

Eclampsia, like preeclampsia, tends to occur more commonly in first pregnancies and young mothers where it is thought that exposure to paternal antigens still has been low. Further, women with preexisting vascular diseases (hypertension, diabetes, and nephropathy) or thrombophilic diseases such as the antiphospholipid syndrome are at higher risk to develop preeclampsia and eclampsia. Conditions with a large placenta (multiple gestation, hydatiform mole) also predispose for toxemia. Further, there is a genetic component; patients whose mother or sister had the condition are at higher risk. Patients with eclampsia are at increased risk for pre-eclampsia-eclampsia in a later pregnancy.

C. IgA Nephropathy

IgA nephropathy (also known as IgA nephritis, IgAN, Berger's disease, and synpharyngitic glomerulonephritis) is a form of glomerulonephritis (inflammation of the glomeruli of the kidney). IgA nephropathy is the most common glomerulonephritis throughout the world. Primary IgA nephropathy is characterized by deposition of the IgA antibody in the glomerulus. There are other diseases associated with glomerular IgA deposits, the most common being Henoch-Schönlein purpura (HSP), which is considered by many to be a systemic form of IgA nephropathy. Henoch-Schönlein purpura presents with a characteristic purpuric skin rash, arthritis, and abdominal pain and occurs more commonly in young adults (16-35 yrs old). HSP is associated with a more benign prognosis than IgA nephropathy. In IgA nephropathy there is a slow progression to chronic renal failure in 25-30% of cases during a period of 20 years.

D. IgM Nephropathy

IgM nephropathy is a condition where deposits of IgM antibody in the glomerulus cause scarring and inflammation within the kidney. Symptoms are variable and in most cases, there are no symptoms, but the damage to the glomeruli causes protein to appear in the urine. Normally this condition is quite painless. However, in some cases there may be some pain over the kidneys, often occurring in attacks after a viral infection.

The cause of IgM nephropathy is unknown. IgM is part of the body's defense against infection. As the antibody travels around in the blood and passes through the kidney it can get deposited in the glomeruli and then can cause an inflammatory reaction.

E. Diabetic Nephropathy

Diabetic nephropathy, also known as Kimmelstiel-Wilson syndrome and intercapillary glomerulonephritis, is a progressive kidney disease caused by angiopathy of capillaries in the kidney glomeruli. It is characterized by nephrotic syndrome and diffuse glomerulosclerosis. It is due to longstanding diabetes mellitus and is a prime cause for dialysis. The earliest detectable change in the course of diabetic nephropathy is a thickening in the glomerulus. At this stage, the kidney may start allowing more serum albumin than normal in the urine. As diabetic nephropathy progresses, increasing numbers of glomeruli are destroyed by nodular glomerulosclerosis and the amount of albumin excreted in the urine increases.

F. Lupus Nephritis

Lupus nephritis is a kidney disorder that is a complication of systemic lupus erythematosus. Lupus nephritis occurs when antibodies and complement build up in the kidneys, causing inflammation. It often causes proteinuria and may progress rapidly to renal failure. Nitrogen waste products build up in the bloodstream. Systemic lupus erythematosus causes various disorders of the internal structures of the kidney, including interstitial nephritis. Lupus nephritis affects approximately 3 out of 10,000 people.

G. Goodpasture's Syndrome

Goodpasture's syndrome (also known as Goodpasture's disease and anti-glomerular basement membrane disease) is a rare condition characterized by glomerulonephritis and hemorrhaging of the lungs. This autoimmune disease is triggered when the patient's immune system attacks Goodpasture antigen, which is found in the kidney and lung, and in time, causing damage to these organs. The kidney portion of the disease mostly affects the glomeruli causing a form of nephritis. It is usually not detected until a rapid advance of the disease occurs and kidney function can be completely lost in a matter of days. Blood spills into the urine causing hematuria, the volume of urine output decreases, and urea and other products usually excreted by the kidney are retained and build up in the blood. Symptoms include loss of appetite and malaise at first and then, when the damage is more advanced, breathlessness, high blood pressure, and edema. The kidney involvement usually presents as nephritic syndrome, i.e., hematuria, a reduced glomerular filtration rate, and high blood pressure. This is in contrast to nephrotic syndrome, a more rare outcome of Goodpasture's, characterized by an abnormally large amount protein in the urine (proteinuria), coupled with severe edema.

H. Membranoproliferative Glomerulonephritis I/II/III

Membranoproliferative glomerulonephritis is a type of glomerulonephritis caused by deposits in the kidney glomerular mesangium and basement membrane thickening, activating complement and damaging the glomeruli. There are three types of membranoproliferative glomerulonephritis. Type I is caused by immune complexes depositing in the kidney and is believed to be associated with the classical complement pathway. Type II is similar to Type I, however, it is believed to be associated with the alternative complement pathway. Type III is very rare and it is characterized by a mixture of subepithelial deposits and the typical pathological findings of Type I disease.

I. Poststreptococcal Glomerulnephritis

Poststreptococcal glomerulnephritis is a common complication of infections, typically Streptococcal pharyngitis and can be a risk factor for future albuminuria. The exact pathology remains unclear, but it is believed to be type III hypersensitivity reaction. Immune complexes become lodged in the glomerular basement membrane. Complement activation leads to destruction of the basement membrane. It has also been proposed that specific antigens from certain nephrotoxic streptococcal infections have a high affinity for basement membrane proteins, giving rise to particularly severe, long lasting antibody response.

J. Progressive (Crescentic) Glomerulonephritis

Progressive (crescentic) glomerulonephritis (PG) is a syndrome of the kidney that, if left untreated, rapidly progresses into acute renal failure and death within months. In 50% of cases, PG is associated with an underlying disease such as Goodpasture's syndrome, systemic lupus erythematosus, or Wegener granulomatosis; the remaining cases are idiopathic. Regardless of the underlying cause, PG involves severe injury to the kidney's glomeruli, with many of the glomeruli containing characteristic crescent-shaped scars. Patients with PG have hematuria, proteinuria, and occasionally, hypertension and edema. The clinical picture is consistent with nephritic syndrome, although the degree of proteinuria may occasionally exceed 3 g/24 hours, a range associated with nephrotic syndrome. Untreated disease may progress to decreased urinary volume (oliguria), which is associated with poor kidney function.

K. Membranous Glomerulonephritis

Membranous glomerulonephritis (MGN) is a slowly progressive disease of the kidney affecting mostly patients between ages of 30 and 50 years, usually Caucasian. It can develop into nephrotic syndrome. MGN is caused by circulating immune complex. Current research indicates that the majority of the immune complexes are formed via binding of antibodies to antigens in situ to the glomerular basement membrane. The said antigens may be endogenous to the basement membrane, or deposited from systemic circulation.

### Measurement of Urine Protein Levels

Protein levels in urine can be measured using methods known in the art. Until recently, an accurate protein measurement required a 24 hour urine collection. In a 24 hour collection, the patient urinates into a container, which is kept refrigerated between trips to the bathroom. The patient is instructed to begin collecting urine after the first trip to the bathroom in the morning. Every drop of urine for the rest of the day is to be collected in the container. The next morning, the patient adds the first urination after waking and the collection is complete.

More recently, researchers have found that a single urine sample can provide the needed information. In the newer technique, the amount of albumin in the urine sample is compared with the amount of creatinine, a waste product of normal muscle breakdown. The measurement is called a urine albumin-to-creatinine ratio (UACR). A urine sample containing more than 30 milligrams of albumin for each gram of creatinine (30 mg/g) is a warning that there may be a problem. If the laboratory test exceeds 30 mg/g, another UACR test should be performed 1 to 2 weeks later. If the second test also shows high levels of protein, the person has persistent proteinuria, a sign of declining kidney function, and should have additional tests to evaluate kidney function.

Tests that measure the amount of creatinine in the blood will also show whether a subject's kidneys are removing wastes efficiently. Too much creatinine in the blood is a sign that a person has kidney damage. A physician can use the creatinine measurement to estimate how efficiently the kidneys are filtering the blood. This calculation is called the estimated glomerular filtration rate, or eGFR. Chronic kidney disease is present when the eGFR is less than 60 milliliters per minute (mL/min).

### TRPC5

TRPC is a family of transient receptor potential cation channels in animals. TRPC5 is subtype of the TRPC family of mammalian transient receptor potential ion channels. Three examples of TRPC5 are highlighted below in Table 1.

TABLE 1
The TRPC5 orthologs from three different species along
with their GenBank Ref Seq Accession Numbers.

| Species | Nucleic Acid | Amino Acid | GeneID |
|---|---|---|---|
| Homo sapiens | NM_012471.2 | NP_036603.1 | 7224 |
| Mus musculus | NM_009428.2 | NP_033454.1 | 22067 |
| Rattus norvegicus | NM_080898.2 | NP_543174.1 | 140933 |

Provided herein are methods for reducing or inhibiting proteinuria in a subject by administering to the subject a therapeutically effective amount of a TRPC5 inhibitor.

### Subjects to be Treated

In one aspect of the methods described herein, a subject is selected on the basis that they have, or are at risk of developing, proteinuria. A subject that has, or is at risk of developing, proteinuria is one having one or more symptoms of the condition. Symptoms of proteinuria are known to those of skill in the art and include, without limitation, large amounts of protein in the urine, which may cause it to look foamy in the toilet. Loss of large amounts of protein may result in edema, where swelling in the hands, feet, abdomen, or face may occur. These are signs of large protein loss and indicate that kidney disease has progressed. Laboratory testing is the only way to find out whether protein is in a subject's urine before extensive kidney damage occurs.

A subject that has, or is at risk of developing, proteinuria is one with diabetes, hypertension, or certain family backgrounds. In the United States, diabetes is the leading cause of end-stage renal disease (ESRD). In both type 1 and type 2 diabetes, albumin in the urine is one of the first signs of deteriorating kidney function. As kidney function declines, the amount of albumin in the urine increases. Another risk factor for developing proteinuria is hypertension. Proteinuria in a person with high blood pressure is an indicator of declining kidney function. If the hypertension is not controlled, the person can progress to full kidney failure. African Americans are more likely than Caucasians to have high blood pressure and to develop kidney problems from it, even when their blood pressure is only mildly elevated. Other groups at risk for proteinuria are American Indians, Hispanics/Latinos, Pacific Islander Americans, older adults, and overweight subjects. These at-risk groups and people who have a family history of kidney disease should have their urine tested regularly.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

### Inhibitors of TRPC5

Examples of inhibitors of TRPC5 include antibodies that bind to and/or inhibit a TRPC5, as well as nucleic acids that inhibit TRPC5 gene expression. Such modulators can be provided as a pharmaceutical composition.

A. Known Inhibitors of TRPC5

Several compounds that target TRPC5 have emerged including 2-aminoethoxydiphenylborane and 1-oleoyl-2-acetyl-sn-glycerol.

B. Antibodies to TRPC5

Antibodies can be produced that bind to TRPC5. For example, an antibody can bind to TRPC5 and prevent TRPC5 activity or Ca²⁺ transport. The term “antibody” as used herein refers to an immunoglobulin molecule or immunologically active portion thereof, i.e., an antigen-binding fragment. Examples of immunologically active portions of immunoglobulin molecules include F(ab′) and F(ab′)₂ fragments, which retain the ability to bind antigen. Such fragments can be obtained commercially, or using methods known in the art. For example, F(ab′)₂ fragments can be generated by treating the antibody with an enzyme such as pepsin, a non-specific endopeptidase that normally produces one F(ab′)₂ fragment and numerous small peptides of the Fc portion. The resulting F(ab′)₂ fragment is composed of two disulfide-connected Fab units. The Fc fragment is extensively degraded and can be separated from the F(ab′)₂ by dialysis, gel filtration or ion exchange chromatography. F(ab′) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50.00 Dalton Fc fragment; to isolate the F(ab′) fragments, the Fc fragments can be removed, e.g., by affinity purification using protein A/G. A number of kits are available commercially for generating F(ab′) fragments, including the ImmunoPure IgG1 Fab and F(ab′)₂ Preparation Kit (Pierce Biotechnology, Rockford, Ill.). In addition, commercially available services for generating antigen-binding fragments can be used, e.g., Bio Express, West Lebanon, N.H.

The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to a Fc receptor, e.g., it has a mutagenized or deleted Fc receptor binding region. The antibody can be coupled to a toxin or imaging agent.

Methods for making suitable antibodies are known in the art. A full-length TRPC5 protein or antigenic peptide fragment thereof can be used as an immunogen, or can be used to identify antibodies made with other immunogens, e.g., cells, membrane preparations, and the like, e.g., E rosette positive purified normal human peripheral T cells, as described in U.S. Pat. Nos. 4,361,549 and 4,654,210.

Methods for making monoclonal antibodies are known in the art. Basically, the process involves obtaining antibody-secreting immune cells (lymphocytes) from the spleen of a mammal (e.g., mouse) that has been previously immunized with the antigen of interest (e.g., a cancer-related antigen) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein (Nature 256:495, 1975), which is hereby incorporated by reference.

Mammalian lymphocytes are immunized by in vivo immunization of the animal (e.g., a mouse) with a cancer-related antigen. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511, 1976, which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected at a total volume of 100:1 per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/kg IV. This and other procedures for raising polyclonal antibodies are disclosed in E. Harlow, et. al., editors, Antibodies: A Laboratory Manual (1988).

In addition to utilizing whole antibodies, the invention encompasses the use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

Chimeric, humanized, de-immunized, or completely human antibodies are desirable for applications which include repeated administration, e.g., therapeutic treatment of human subjects.

Chimeric antibodies generally contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999, 1987). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc Natl Acad Sci USA 81:6801, 1984; Morrison and Oi, Adv Immunol 44:65, 1988) (which preserves the ligand-binding properties, but which also retains the immunogenicity of the nonhuman variable domains); (b) by grafting only the nonhuman CDRs onto human framework and constant regions with or without retention of critical framework residues (Jones et al., Nature 321:522, 1986; Verhoeyen et al., Science 239:1539, 1988); or (c) by transplanting the entire nonhuman variable domains (to preserve ligand-binding properties) but also “cloaking” them with a human-like surface through judicious replacement of exposed residues (to reduce antigenicity) (Padlan, Molec Immunol 28:489, 1991).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323, 1988; Queen et al., Proc Natl Acad Sci USA 86:10,029, 1989). The framework residues which need to be preserved can be identified by computer modeling. Alternatively, critical framework residues may potentially be identified by comparing known antibody combining site structures (Padlan, Mol Immun 31(3):169-217, 1994). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525, 1986).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann NY Acad Sci 880:263-80, 1999; and Reiter, Clin Cancer Res 2:245-52, 1996). The single chain antibody can be dimerized or multimerized to generate multivalent antibodies having specificities for different epitopes of the same target protein. In some embodiments, the antibody is monovalent, e.g., as described in Abbs et al., Ther Immunol 1(6):325-31, 1994, incorporated herein by reference.

TRPC5 antibodies are also commercially available, e.g., from Abcam, Novus Biologicals, Thermo Scientific Pierce Antibodies, and Sigma-Aldrich. These antibodies can be modified as known in the art and disclosed herein, e.g., humanized or deimmunized.

C. TRPC5 Inhibitory Nucleic Acids

Nucleic acid molecules (e.g., RNA molecules) can be used to inhibit (i.e., reduce) TRPC5 expression or activity. A TRPC5 inhibitor can be a siRNA, antisense RNA, a ribozyme, or aptamer, which can specifically reduce the expression of TRPC5. In some aspects, a cell or subject can be treated with a compound that reduces the expression of TRPC5. Such approaches include oligonucleotide-based therapies such as RNA interference, antisense, ribozymes, and aptamers.

i. siRNA Molecules

RNA interference (RNAi) is a process whereby double-stranded RNA (dsRNA, also referred to herein as siRNAs or ds siRNAs, for double-stranded small interfering RNAs) induces the sequence-specific degradation of homologous mRNA in animals and plant cells (Hutvagner and Zamore, Curr Opin Genet Dev 12:225-232, 2002; Sharp, Genes Dev 15:485-490, 2001). In mammalian cells, RNAi can be triggered by 21-nucleotide (nt) duplexes of small interfering RNA (siRNA) (Chiu et al., Mol Cell 10:549-561, 2002; Elbashir et al., Nature 411:494-498, 2001), or by micro-RNAs (miRNA), functional small-hairpin RNA (shRNA), or other dsRNAs which are expressed in vivo using DNA templates with RNA polymerase III promoters (Zeng et al., Mol Cell 9:1327-1333, 2002; Paddison et al., Genes Dev 16:948-958, 2002; Lee et al., Nature Biotechnol 20:500-505, 2002; Paul et al., Nature Biotechnol 20:505-508, 2002; Tuschl, Nature Biotechnol 20:440-448, 2002; Yu et al., Proc Natl Acad Sci USA 99(9):6047-6052, 2002; McManus et al., RNA 8:842-850, 2002; Sui et al., Proc Natl Acad Sci USA 99(6):5515-5520, 2002).

The nucleic acid molecules or constructs can include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can transcribed in vitro from a DNA template, or in vivo from, e.g., shRNA. The dsRNA molecules can be designed using any method known in the art; a number of algorithms are known, and are commercially available. Gene walk methods can be used to optimize the inhibitory activity of the siRNA.

The nucleic acid compositions can include both siRNA and modified siRNA derivatives, e.g., siRNAs modified to alter a property such as the pharmacokinetics of the composition, for example, to increase half-life in the body, as well as engineered RNAi precursors.

siRNAs can be delivered into cells by methods known in the art, e.g., cationic liposome transfection and electroporation. siRNA duplexes can be expressed within cells from engineered RNAi precursors, e.g., recombinant DNA constructs using mammalian Pol III promoter systems (e.g., H1 or U6/snRNA promoter systems (Tuschl 2002, supra) capable of expressing functional double-stranded siRNAs; (Bagella et al., J Cell Physiol 177:206-213, 1998; Lee et al., 2002, supra; Miyagishi et al., 2002, supra; Paul et al., 2002, supra; Yu et al., 2002, supra; Sui et al., 2002, supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998, supra; Lee et al., 2002, supra; Miyagishi et al., 2002, supra; Paul et al., 2002, supra; Yu et al., 2002, supra; Sui et al., 2002, supra). Constructs containing siRNA sequence under the control of T7 promoter also make functional siRNAs when cotransfected into the cells with a vector expression T7 RNA polymerase (Jacque, 2002, supra).

ii. Antisense Nucleic Acids

An “antisense” nucleic acid can include a nucleotide sequence that is complementary to a “sense” nucleic acid encoding a protein, e.g., complementary to the coding strand of a double-stranded cDNA molecule or complementary to a TRPC5 mRNA sequence. The antisense nucleic acid can be complementary to an entire coding strand of a target sequence, or to only a portion thereof. In another embodiment, the antisense nucleic acid molecule is antisense to a “noncoding region” of the coding strand of a nucleotide sequence (e.g., the 5′ and 3′ untranslated regions).

An antisense nucleic acid can be designed such that it is complementary to the entire coding region of a target mRNA, but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

In some embodiments, the antisense nucleic acid molecule is an ∀V-anomeric nucleic acid molecule. An ∀-anomeric nucleic acid molecule forms specific double-stranded hybrids with complementary RNA in which, contrary to the usual β-units, the strands run parallel to each other (Gaultier et al., Nucleic Acids Res 15:6625-6641, 1987). The antisense nucleic acid molecule can also comprise a 2′-o-methylribonucleotide (Inoue et al., Nucleic Acids Res 15:6131-6148, 1987) or a chimeric RNA-DNA analogue (Inoue et al., FEBS Lett 215:327-330, 1987).

In some embodiments, the antisense nucleic acid is a morpholino oligonucleotide (see, e.g., Heasman, Dev Biol 243:209-14, 2002; Iversen, Curr Opin Mol Ther 3:235-8, 2001; Summerton, Biochim Biophys Acta 1489:141-58, 1999).

Target gene expression can be inhibited by targeting nucleotide sequences complementary to a regulatory region (e.g., promoters and/or enhancers) to form triple helical structures that prevent transcription of the Spt5 gene in target cells. See generally, Helene, Anticancer Drug Des 6:569-84, 1991; Helene, Ann NY Acad Sci 660:27-36, 1992; and Maher, Bioassays 14:807-15, 1992. The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

iii. Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target nucleic acid can include one or more sequences complementary to the nucleotide sequence of a cDNA described herein, and a sequence having known catalytic sequence responsible for mRNA cleavage (see U.S. Pat. No. 5,093,246 or Haselhoff and Gerlach, Nature 334:585-591, 1988). For example, a derivative of a Tetrahymena L-19 IVS RNA can be constructed in which the nucleotide sequence of the active site is complementary to the nucleotide sequence to be cleaved in a target mRNA. See, e.g., Cech et al., U.S. Pat. No. 4,987,071; and Cech et al., U.S. Pat. No. 5,116,742. Alternatively, a target mRNA can be used to select a catalytic RNA having a specific ribonuclease activity from a pool of RNA molecules. See, e.g., Bartel and Szostak, Science 261:1411-1418, 1993.

iv. Aptamers

Aptamers are short oligonucleotide sequences which can specifically bind specific proteins. It has been demonstrated that different aptameric sequences can bind specifically to different proteins, for example, the sequence GGNNGG where N=guanosine (G), cytosine (C), adenosine (A) or thymidine (T) binds specifically to thrombin (Bock et al., Nature 355:564-566, 1992; and U.S. Pat. No. 5,582,981, Toole et al., 1996). Methods for selection and preparation of such RNA aptamers are known in the art (see, e.g., Famulok, Curr Opin Struct Biol 9:324, 1999; Herman and Patel, J Sci 287:820-825, 2000; Kelly et al., J Mol Biol 256:417, 1996; and Feigon et al., Chem Biol 3:611, 1996).

### Administration of Inhibitory Nucleic Acid Molecules

The inhibitory nucleic acid molecules described herein can be administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, inhibitory nucleic acid molecules can be modified to target selected cells and then administered systemically. For systemic administration, inhibitory nucleic acid molecules can be modified such that they specifically bind to receptors or antigens expressed on a selected cell surface, e.g., by linking the inhibitory nucleic acid molecules to peptides or antibodies that bind to cell surface receptors or antigens. The inhibitory nucleic acid molecules can also be delivered to cells using the vectors described herein. To achieve sufficient intracellular concentrations of the inhibitory nucleic acid molecules, vector constructs in which the inhibitory nucleic acid molecule is placed under the control of a strong promoter can be used.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

### Methods of Screening (Test Compounds)

Included herein are methods for screening test compounds, e.g., polypeptides, polynucleotides (including inhibitory nucleic acids), inorganic or organic large or small molecule test compounds, to identify agents useful in the treatment of proteinuria.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6, 1997). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a cell, and one or more effects of the test compound is evaluated. In some embodiments, a cultured or primary cell for example, the ability of the test compound to inhibit calcium transport can be evaluated. In other embodiments, a cultured or primary cell for example, the ability of the test compound to inhibit TRPC5 expression can be evaluated, e.g., assay TRPC5 mRNA or protein levels.

In some embodiments, the test sample is, or is derived from (e.g., a sample taken from) an in vivo model of a disorder as described herein. For example, an animal model, e.g., a rodent such as a rat, can be used.

Methods for evaluating these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern Genetic Analysis, 1999,W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on TRPC5.

A test compound that has been screened by a method described herein and determined to inhibit calcium transport can be considered a candidate compound. A candidate compound that has been screened, e.g., in an in vivo model of a disorder, e.g., proteinuria, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that inhibit calcium transport) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating proteinuria. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a disorder associated with proteinuria, as described herein. The animal can be monitored for a change in the disorder, e.g., for an improvement in a parameter of the disorder, e.g., a parameter related to clinical outcome. In some embodiments, the parameter is urine protein level, and an improvement would be 0-10 mg/dl. In some embodiments, the subject is a human and the parameter is urine protein level.

The following are examples of the practice of the invention.

### EXAMPLES

The invention is further described in the following examples, which do not limit the scope of the invention described in the claims.

Materials. All the reagents used in this study, unless otherwise stated, were from Sigma-Aldrich (St. Louis, Mo.).

Podocyte cell culture. Wild-type mouse podocytes (passages 7 to 18) derived from the Immortomouse stably expressing the temperature-sensitive SV40 T-antigen were generously provided by Peter Mundel (University of Miami School of Medicine, FL) and cultured as previously described (Asanuma et al., Nat Cell Biol 8, 485-491, 2006). Briefly, podocytes were plated on collagen-coated cell culture dishes (100 μg/ml Type I Collagen, Gibco Invitrogen) and maintained at 33° C. in a humidified atmosphere of 5% CO₂ in RPMI1640 medium supplemented with 10% heat-inactivated FBS, 100 U/ml Penicillin, 100 μg/ml streptomycin, and 100 U/ml Interferon-γ (IFN-γ). Under these permissive culture conditions, the podocytes remain in the undifferentiated, replication-competent state. Podocytes were thermoshifted to 38° C. and maintained in IFN-γ-free culture medium to induce growth arrest and differentiation. Podocyte-specific markers for differentiation are expressed within 7 days of culture in these non-permissive conditions. Immortalized rat podocytes stably expressing the AngII Type 1 Receptor were cultured in RPMI 1640 supplemented with 250 mg/ml G418 as described above.

Lentivirus construction and shRNA-mediated gene silencing. Podocytes were exposed to lentivirus encoding validated shRNA sequences (Open Biosystems) for 24 hours in cell culture medium supplemented with 4 μg/ml polybrene (Chemicon International). For all experiments, parallel infections with the enhanced GFP virus in the VVPW vector, scrambled shRNA, or polybrene alone were used as control. All experiments were performed at least 48 hours post-infection of podocytes that were thermoshifted 5-7 days prior to the assay. Lentiviral particles pseudotyped with the vesicular stomatitis virus glycoprotein (VSV-G) were constructed according to the manufacturer's protocol. Briefly, low-passage HEK293T cells were transfected with the pLKO.1 plasmid encoding the shRNA, the packaging plasmid pMDG.2 ΔR8.91, and the envelope plasmid pCMV-VSV-G at a ratio of 10:10:1 using the Minis Trans-IT LT-1 transfection reagent. Enhanced GFP encoded in the pLVX vector was used to assess transfection efficiency. The medium was replaced with the virus harvest media 18 hours post-transfection, and viral particles were collected twice at 24 hour intervals thereafter. The TRPC5 and TRPC6 shRNA consisted of three pooled shRNA constructs from the RNAi Consortium library of The Broad Institute that were screened on the basis of knockdown efficiency. The TRPC5 hairpin sequences are:

CCGGCCTGGCAACTATTTCCCTGAACTCGAGTTCAGGGAAATAGTTGCC
AGGTTTTTG,
CCGGGCAATCAAATACCACCAGAAACTCGAGTTTCTGGTGGTATTTGAT
TGCTTTTTG,
CCGGCCCAAGTCATTTCTATACCTTCTCGAGAAGGTATAGAAATGACTT
GGGTTTTTG.

The TRPC6 hairpin sequences are

CCGGGCTCATTATATCCTGGGTAATCTCGAGATTACCCAGGATATAATG
AGCTTTTTG,
CCGGCGTTCTGTATGGTGTCTATAACTCGAGTTATAGACACCATACAGA
ACGTTTTTG,
CCGGCGTCCAAATCTCAGCCGTTTACTCGAGTAAACGGCTGAGATTTGG
ACGTTTTTG.

Viral stocks (<1×10⁶ Transducing Units/ml) were used at a multiplicity of infection of 5-7.5. For overexpression studies, lentivirus particles encoding wild-type or dominant-negative TRPC6 VVPW plasmid were produced in HEK293T by co-transfection with the packaging and envelope plasmids at a ratio of 3:2:1.

RT-PCR. Total RNA was extracted from podocytes using the Aurum miniprep Kit (Bio-Rad). mRNA was reverse transcribed using the Omniscript RT (Qiagen) with oligo dT primers. Expression levels of the TRPC channels were detected by 3-step PCR using two independent sets of sequence-specific forward and reverse oligos. PCR products were obtained with the Phusion DNA polymerase (Finnzymes) at the optimized thermocycling conditions (40 cycles of 98° C. 5 seconds denaturation, 69° C. 30 seconds annealing, 72° C. 30 seconds extension) and visualized in a 1.2% agarose gel. Transcript levels for β-actin were assayed in parallel as control.

Western Blot and GTPase activation assay. Serum-deprived cells were exposed to AngII for three hours and washed twice with PBS. Cells were scraped into ice-cold RIPA buffer supplemented with protease inhibitors (Roche) and the phosphatase blockers NaF, Na₃VO₄, and lysed on ice for 30 minutes. Lysates were clarified by centrifugation at 13,000g for 15 minutes, 4° C. and the total protein content was measured by BCA assay (Pierce). Total protein (25 μg) was resolved in pre-cast 4-12% Bis-Tris gels (Invitrogen) and electrotransfered to PVDF membranes. Membranes were probed with primary antibodies to Rac1 or RhoA (Cell Biolabs, CA), GFP (Santa Cruz, Calif.) or β-actin. For small GTPase activation assays, 500 μg of total protein from HEK293T lysates was incubated with Rhotekin- or PAK1-conjugated agarose beads (Cell Biolabs, CA) for 1 hour at 4° C. Activated, GTP-bound forms of Rho, Rac1, and Cdc42 were recovered in lysis buffer by boiling, and resolved in Bis-Tris gels as described above.

Immunocytochemistry, wound healing assays, and confocal imaging. Podocytes were seeded in collagen-coated coverslips (12 mm) to 80% confluence and infected with lentivirus as described above. The cells were washed twice with ice-cold PBS and fixed with 5% paraformaldehyde (Electron Microscopy Sciences, PA) in PBS for 1 hr before permeabilization 0.2% Triton X-100. For wound healing assays, podocytes or Swiss 3T3 fibroblasts cultured in monolayers were serum-starved for 3 hours before an artificial wound was made with a sterile 100 μL polypropylene pipette tip (2 μm outer diameter). Cells were either directly fixed (t=0) or replaced in complete medium for migration assays before fixation at various time points (36 hours, 100 hours post-scratch). Nonspecific immunoreactivity was blocked with 3% BSA. For double immunostaining, podocytes were incubated with antibodies to Paxillin (Chemicon International) and detected with Alexa-488-conjugated secondary antibodies (Molecular Probes). Actin structures were labeled with Alexa-564-conjugated Phalloidin. Pharmacologic agents used: Losartan (200 nM), Y27632 (10 μM). Confocal images were acquired with a Zeiss upright confocal microscope. Images from an optical slice of ˜4-5 μm were acquired at a resolution of 600 ppi using Zeiss Pascal software. Image analysis was performed in ImageJ or Adobe Photoshop for Mac OS X.

Statistical Analysis. Statistical significance was evaluated by One-Way ANOVA with Fisher's LSD test, unless otherwise stated. P<0.05 was considered significant. Values reported as mean±s.e.m.

Calcium imaging. The non-ratiometric, cell permeable Ca²⁺ indicator Fluo-3 (Molecular Probes) was used in all experiments. Fluo-3 (2 μM) was loaded for 20 minutes at 37° C. in podocytes grown to 90% confluence on collagen-coated coverslips. Podocytes were serum starved for 3 hours and the Ca²⁺ indicator was subsequently loaded directly into serum free media. After the 20 minute incubation, cells were washed ×3 with PBS, and the coverslip was loaded onto the recording chamber in the presence of extracellular bath solution containing 2 mM CaCl₂ (identical to the bath solution used in whole cell electrophysiology experiments). Images were acquired in 2 second intervals. Cells were stimulated by the application of 500 nM Angiotensin II to the bath solution at the start of each experiment. Fluorescence intensity was normalized to maximal fluorescence obtained at the end of each experiment by the application of 100 μM Ionomycin, based on the formula F=(Fₘₐₓ (angiotensin)−Fₘᵢₙ)/(Fₘₐₓ (ionomycin)−Fₘᵢₙ). Measurements were analyzed using Zeiss Pascal Software and Microsoft Xcel, and images were prepared in Adobe Photoshop and NIH Image J.

Electrophysiology. Patch-clamp electrophysiology (Axopatch 200B amplifier, Axon Instruments, CA) was performed in the whole-cell configuration. Briefly, fully differentiated podocytes were plated on glass coverslips at low density and placed in the recording chamber. The patch pipettes with resistances of 3-4 MΩ were pulled from borosilicate glass with a P-97 puller (Sutter Instrument) and filled with a solution containing (in mM): 135 CH₃SO₃Cs, 10 CsCl, 3 MgATP, 0.2 NaGTP, 0.2 EGTA, 0.13 CaCl₂, and 10 HEPES, pH 7.3 with CsOH. The bath solution contained (in mM): 135 CH₃SO₃Na, 5 CsCl, 2 CaCl₂, 1 MgCl₂, 10 HEPES, and 10 Glucose, pH 7.4 with NaOH. Angiotensin (500 nM) was applied to the bath solution. Whole-cell currents were recorded from −100 mV to 100 mV voltage ramps over 150 ms and a holding potential of zero. For single channel recordings in the outside-out configuration, we utilized a voltage step protocol from −100 mV to +100 mV delivered at 20 mV intervals and a holding potential of −60 mV. Average pipette resistance filled with pipette solution was 5-10 MΩ. Data were sampled at 10 kHz and filtered at 5 kHz. Single channel data were further off-line filtered at 500 Hz before analysis. In single channel traces, currents were idealized using a manually defined amplitude criterion to assign ion channel opening and closing transitions. Ensemble averages were expressed as open channel probabilities (average current divided by unitary current amplitude and # of channels in each patch) and plotted as histograms. All data were acquired at room temperature and analyzed using pClamp 10 (Axon Instruments, CA).

### Example 1

### TRPC5 and TRPC6 Channels Mediate AngII-Induced Calcium Influx into Podocytes

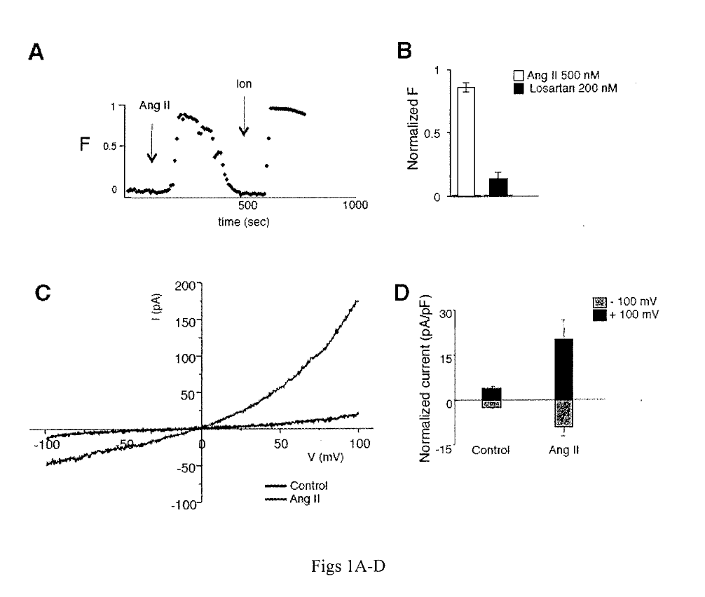

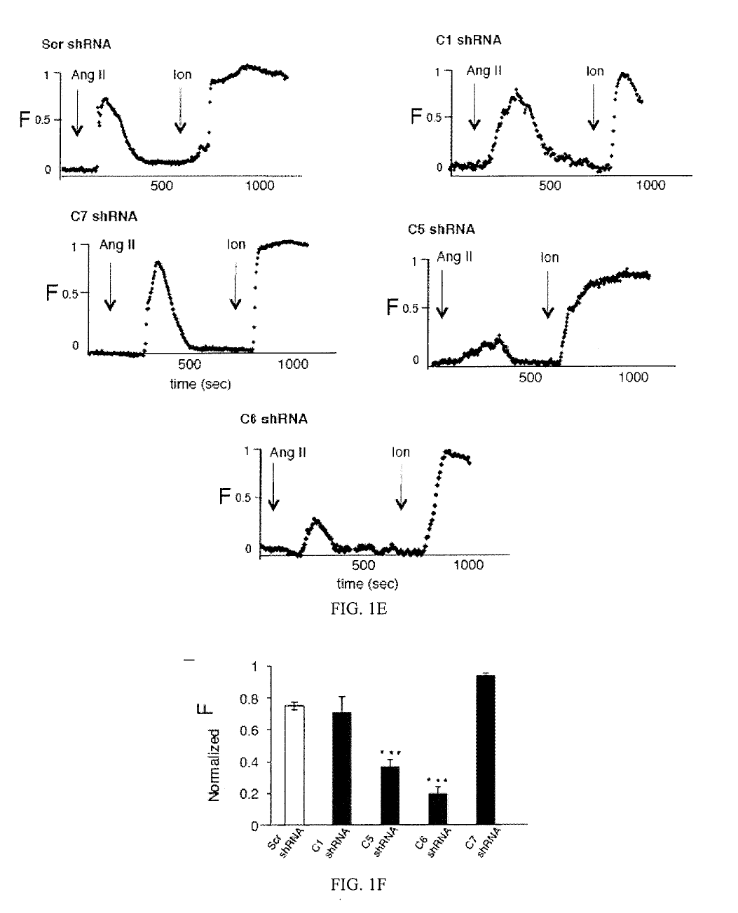

A unifying property for TRPC channels is their activation through G_{q} protein-coupled receptors (GPCRs) (Ramsey et al., Annu Rev Physiol 68, 619-647, 2006). The Angiotensin Type 1 Receptor (AT1R) was identified as a pertinent upstream activator of TRPCs. Wild-type podocytes have a low density of AT1Rs at baseline (Hsu et al., J Mol Med 86, 1379-1394, 2008), and they were thus capable of only occasional, statistically unreliable global Ca²⁺ transients in response to AngII. Stimulation of podocytes expressing the AT1R with AngII (500 nM) resulted in robust Ca²⁺ transients (FIG. 1A). The AT1R antagonist Losartan (200 nM) diminished the amplitude of the Ca²⁺ transient by more than 70% (FIG. 1B). Patch clamp electrophysiology revealed that in the whole cell configuration, application of AngII (500 nM) evoked a current with reversal potential at 0 mV, a small inward component, and a steep outward rectification at positive potentials (FIG. 1C). Although TRPV and TRPM currents could not be excluded, this current-voltage (I-V) relationship was consistent with previously recorded TRPC currents (Clapham, Nature 426, 517-524, 2003). The normalized peak current was 10±4 pA/pF at −100 mV and 20±7 pA/pF at +100 mV (FIG. 1D).

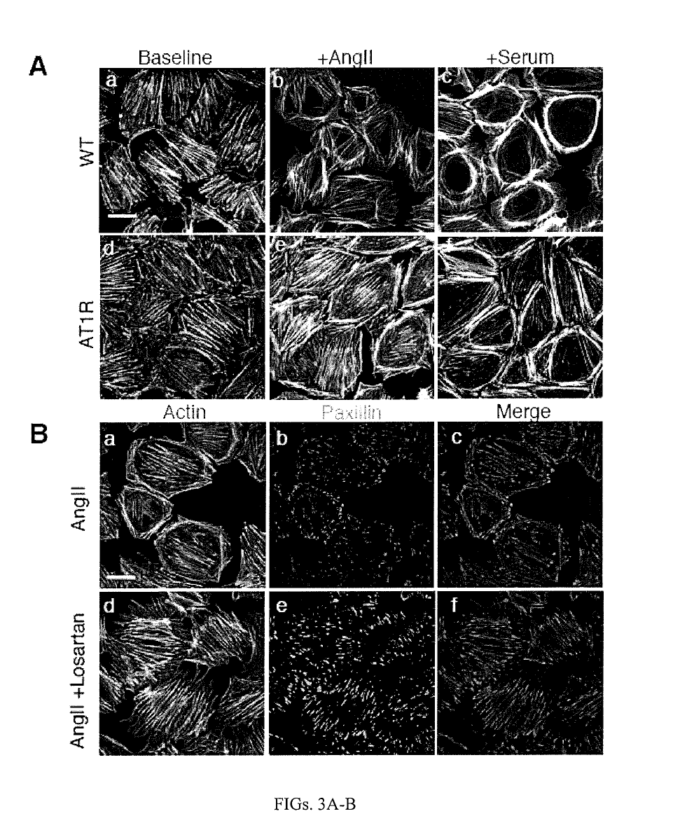

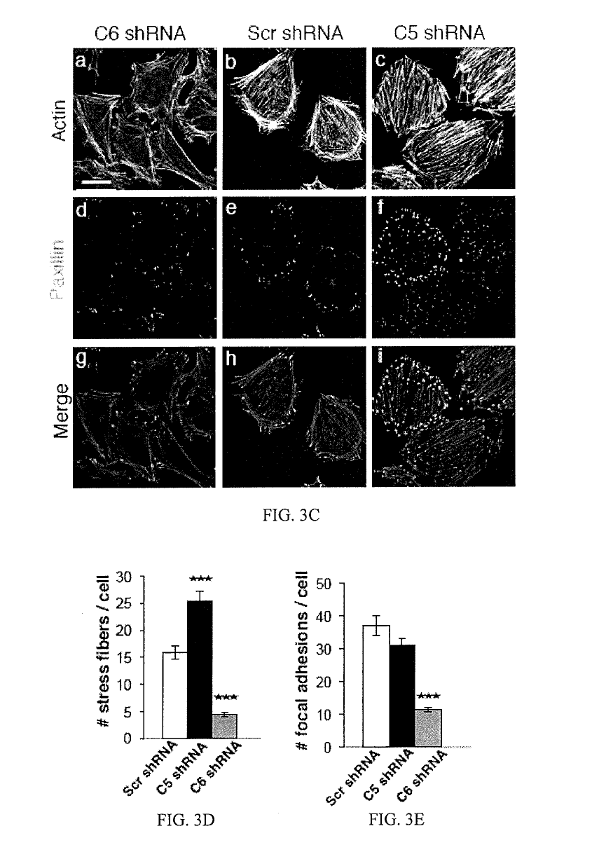

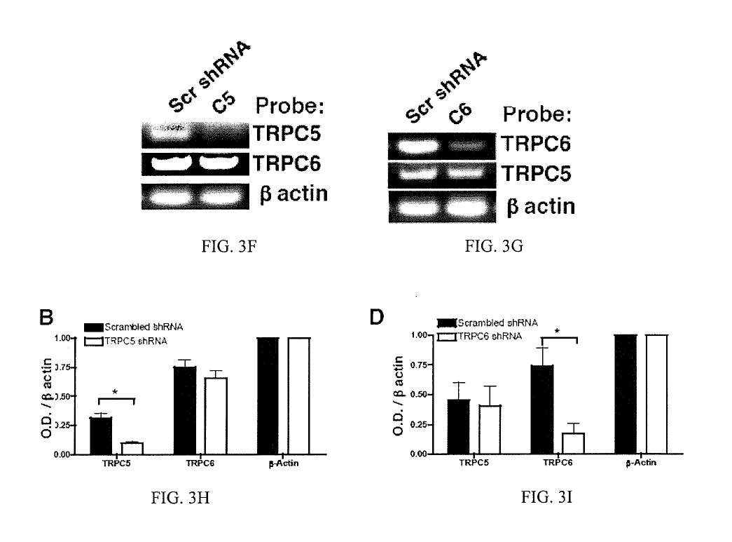

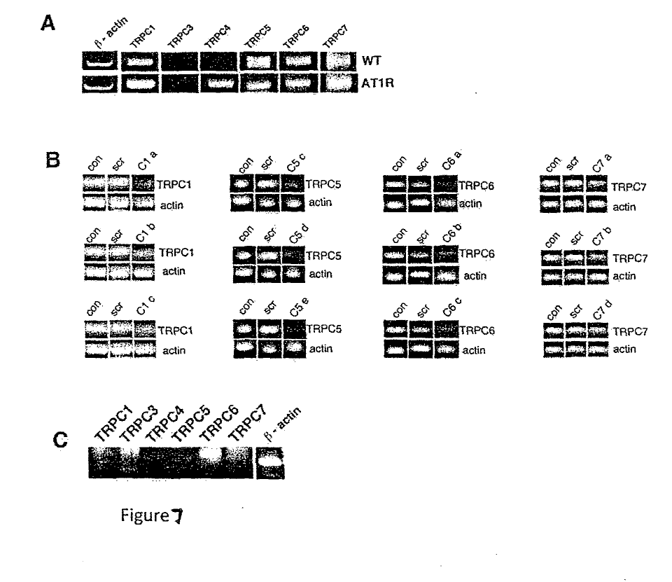

To investigate the molecular identity of this AngII-evoked current, RT-PCR in wild type and AT1R podocytes was performed. Consistent with previous findings Reiser et al., Nat Genet 37, 739-744, 2005), RT-PCR analysis revealed expression of TRPC1, TRPC5, and TRPC6 transcripts in wild-type and AT1R podocytes (FIG. 7A). Additionally, modest expression of the TRPC7 transcript was detected (FIG. 7A). A gene silencing approach was pursued to identify the channels contributing to the recorded TRPC-like current. Using a lentiviral system, shRNA was delivered for each TRPC channel in podocytes with 80-90% knockdown efficiency by RT-PCR analysis (FIG. 7B, also FIGS. 3F-I). Gene silencing of channel subunits essential for AngII-evoked Ca²⁺ influx into podocytes should result in a significant reduction in the peak amplitude of the Ca²⁺ transients compared to controls. In Ca²⁺ imaging experiments, TRPC5 and TRPC6 shRNA reduced the amplitude of the Ca²⁺ transient by 60% and 80%, respectively, as compared to scrambled shRNA controls, TRPC1, or TRPC7 shRNA (FIGS. 1E and 1F). Interestingly, previous studies have shown that in the absence of extracellular Ca²⁺, the AngII-evoked response attributed to internal store release is minimal (less than 10-20% of Fₘₐₓ) compared to control transients measured in extracellular solution containing 2 mM [Ca²⁺] (Hsu et al., J Mol Med 86, 1379-1394, 2008), which was confirmed in the present studies. This suggests that TRPC6 gene silencing largely abrogates the rise in [Ca²⁺]i contributed by ion channels, with the remaining 20% of the [Ca²⁺]i rise contributed by Ca²⁺ release from internal stores and residual TRPC activity. By contrast, TRPC5 gene silencing was less efficient at reducing the amplitude of the transient (60% of Fₘₐₓ), suggesting that this channel makes a smaller contribution to the overall influx of Ca²⁺ through the cell membrane (FIG. 1F). Simultaneous TRPC5 and TRPC6 gene silencing rendered cells nonviable (data not shown), suggesting that the interplay between these two channels plays an essential role in podocyte homeostasis. Taken together, these data point to TRPC5 and TRPC6, but not TRPC1 or TRPC7, as the likely pore forming subunits that contribute to AngII-evoked Ca²⁺ influx in podocytes. Since TRPC5 subunits are not likely to oligomerize with TRPC6 (Strubing et al., J Biol Chem 278, 39014-39019, 2003), these results suggest that in podocytes, the putative Ca²⁺ conduction pores contain homomeric TRPC5 and TRPC6 channels.

### Example 2

### Single Channel Recordings Reveal Distinct TRPC5-Like and TRPC6-Like Conductances in Podocytes

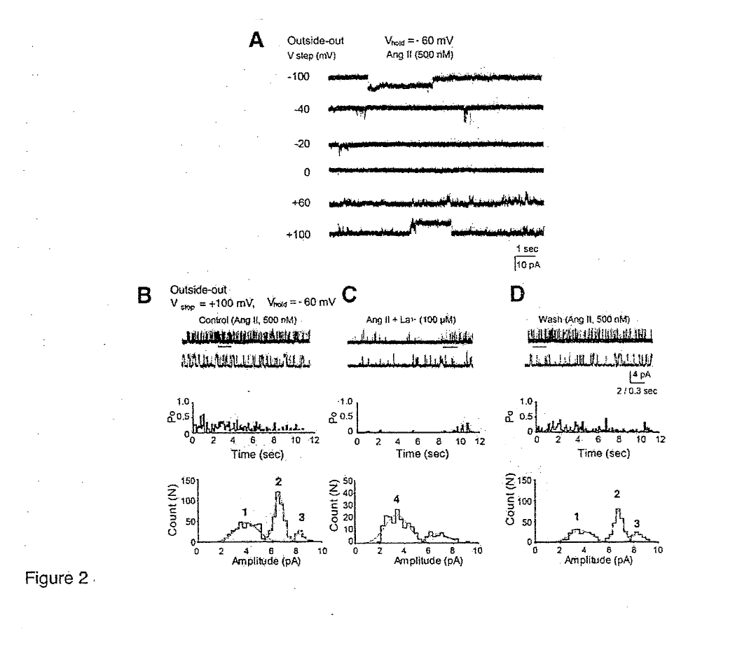

To identify TRPC channels at the single channel level, recordings in the outside-out configuration were performed. A step voltage protocol after AngII bath perfusion revealed native TRPC-like channels with small amplitude current at negative potentials, reversal of the current to the outward direction at 0 mV, and significantly increased amplitude at positive potentials (FIG. 2A). Application of AngII at Vₛₜₑₚ+100 mV revealed three populations of channels with the corresponding single channel conductances: [1] 39 pS, [2] 68 pS, and [3] 80 pS (FIG. 2B). Given that TRPC5 and TRPC6 channels have virtually identical single channel conductances (TRPC5 variably reported as 25-38 pS and TRPC6 variably reported as 25-44 pS (Ramsey et al., Annu Rev Physiol 68, 619-647, 2006; Hofmann et al., Nature 397, 259-263, 1999, Strubing et al., Neuron 29, 645-655, 2001), Lanthanum (La³⁺) was applied to take advantage of its properties as a TRPC6 blocker (Ramsey et al., Annu Rev Physiol 68, 619-647, 2006; Clapham, Nature 426, 517-524, 2003), but a TRPC5 potentiator (Jung et al., J Biol Chem 278, 3562-357, 2003). Application of La³⁺ (100 μM) unmasked a distinct conductance [4] at 33 pS. At the same time, conductances [2] and [3] were abolished (FIG. 2C). Removal of La³⁺ from the bath solution restored conductances [1], [2], and [3] (FIG. 2D). These data suggest that in a representative patch (n=5), the three single channel conductances correspond to a population of active channels composed of a single TRPC5 channel and two TRPC6 channels. In response to AngII, one channel, either TRPC5 or TRPC6, opens, resulting in an average conductance of [1] 39 pS. When TRPC5 and TRPC6 are open simultaneously, their current summation yields conductance [2] of 68 pS. Finally, conductance [3] is mediated by the summation of two TRPC6 channels (FIG. 2B). Application of La³⁺ unmasks the TRPC5 conductance [4] at 33 pS, and blocks all TRPC6 channel conductances (FIG. 2C). Removal of La³⁺ restores the conductances back to baseline (FIG. 2D). These single channel recordings constitute a significant insight into native TRPC5 and TRPC6-like conductances in podocytes.

### Example 3

### TRPC5 and TRPC6 Mediate Distinct Actin Phenotypes

Given that AngII-mediated Ca²⁺ influx has been previously linked to cytoskeletal changes (Asanuma et al., Nat Cell Biol 8, 485-491, 2006; Hsu et al., J Mol Med 86, 1379-1394, 2008), the specific effects of TRPC5 and TRPC6 activity on actin cytoskeleton were examined. To isolate the effects of AT1R-induced TRPC modulation of the cytoskeleton, and to eliminate putative cell culture medium effects, podocytes were serum-starved for 3 hours prior to exposure to AngII or serum. At baseline, both wild-type and AT1R podocytes displayed prominent stress fibers, as previously described (Asanuma et al., Nat Cell Biol 8, 485-491, 2006) (FIG. 3A, a and d). Exposure of wild-type podocytes to AngII resulted in a significant decrease in stress fibers and the reorganization of actin into cortical rings or arcs (FIG. 3A, b) (Hsu et al., J Mol Med 86, 1379-1394, 2008). This phenotype was more prominent in serum-treated controls, indicating that a combination of growth factors present in serum activates pathways that collectively modulate podocyte actin organization (FIG. 3A, c). The observed actin arcs were consistent with previously described structures in many cell types including AT1R cells, where they correlated with a motile cell phenotype (Hsu et al., J Mol Med 86, 1379-1394, 2008). This was confirmed by treating AT1R cells with AngII, which induced prominent actin arc formation (FIG. 3A, d-f). Next, we treated cells with Losartan (200 nM) prior to AngII exposure, which induced robust stress fiber formation (FIG. 3B, d-f vs. a-c). Immunostaining for Paxillin, a marker for focal adhesions, revealed an increase in focal adhesions in Losartan-treated podocytes compared to AngII-treated controls (FIG. 3B, b vs. e).

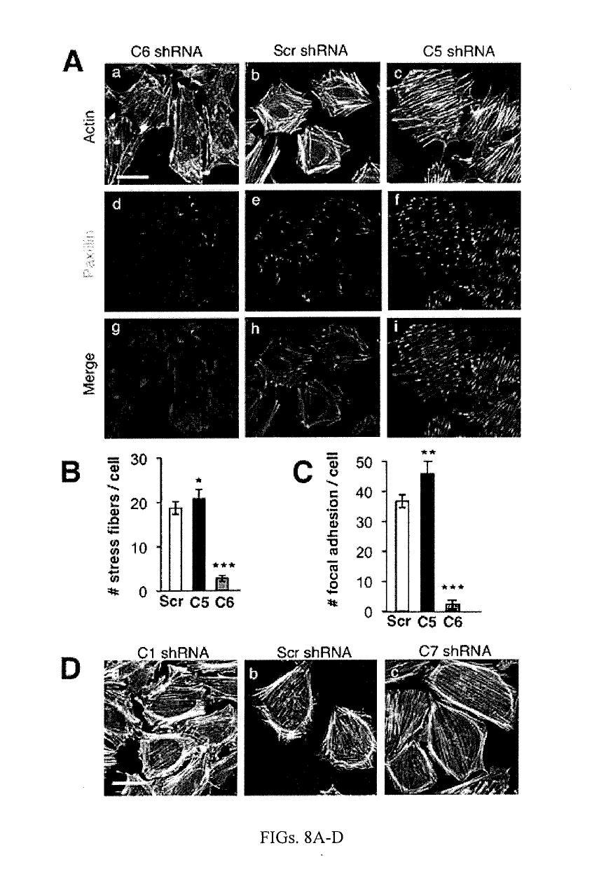

TRPC5- and TRPC6-depleted podocytes were examined for changes to the actin cytoskeleton in response to AngII treatment. Gene silencing of TRPC6 (C6 shRNA) in AT1R and wild-type podocytes resulted in a dramatic loss of stress fibers and AngII-induced actin arcs (AT1R: 15±2 vs. 5±1; WT: 18±2 vs. 4±1; one way ANOVA Fisher's LSD, P<0.001, n=20) (FIGS. 3C, a, g; 3D; 8A and 8B). Paxillin staining was also significantly reduced in TRPC6 depleted cells, indicating a reduction in focal adhesions (AT1R: 30±4 vs. 10±1; WT: 35±5 vs. 5±2) (FIGS. 3C, d; 3E; 8A and 8C). In contrast, gene silencing of TRPC5 (C5 shRNA) in AT1R and wild-type podocytes restored stress fibers (AT1R: 15±2 vs. 25±4; WT: 18±2 vs. 21±2; one way ANOVA Fisher's LSD, P<0.05-0.001) (FIG. 3C, c-i; 3D; 8A and 8B). TRPC5 depleted cells also restored focal adhesions located throughout the cell compared to TRPC6 depleted cells (AT1R: 10±1 vs. 45±5; WT: 5±2 vs. 35±2; One way ANOVA Fisher's LSD, P<0.01) (FIG. 3C, f, I; 3E; 8A and 8C). Gene silencing of TRPC1 or TRPC7 (C1 shRNA or C7 shRNA) had no effect on the actin cytoskeleton, as compared to control cells expressing scrambled shRNA (FIG. 8D). RT-PCR analysis showed that neither TRPC5 nor TRPC6 transcript levels were significantly changed upon silencing of the counterpart channel (FIG. 3F-I), suggesting that the observed phenotypes were not related to upregulation of one channel in the absence of another, as shown previously for TRPC channels in other experimental settings Freichel et al., J Physiol 567, 59-66, 2005). These data suggest that endogenous TRPC6 maintains a contractile, stationary cell phenotype, whereas TRPC5 suppresses stress fiber and focal adhesion formation, promoting a motile phenotype.

### Example 4

### Functional Coupling Between TRPCs and Rho GTPases Regulates the Actin Cytoskeleton

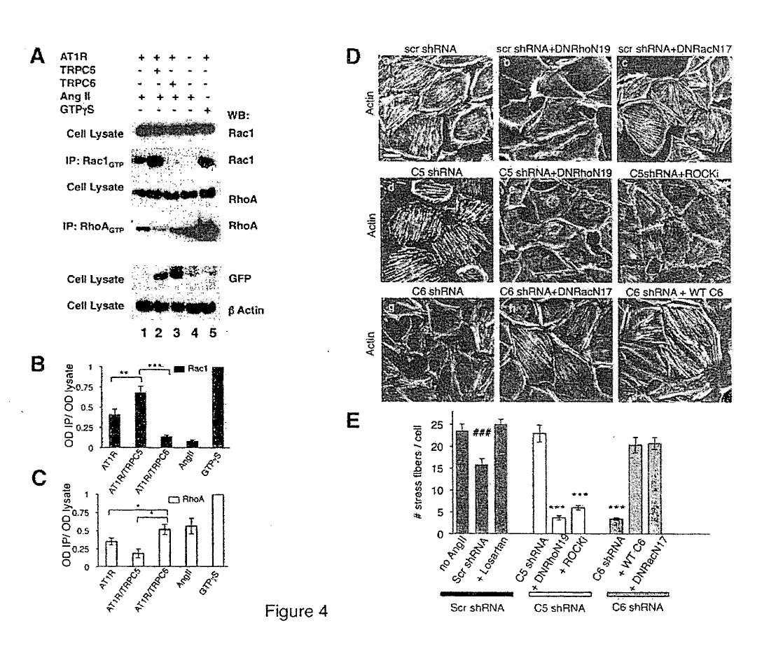

Given the observed contractile and motile cell phenotypes, studies were performed to determine whether TRPC5 and TRPC6 are differentially coupled to signal transduction pathways that maintain a balance between contractility and motility. RhoA enhances contractility and, through a mechanism of reciprocal inhibition, Rac1 enhances motility, both contributing to the delicate balance of cytoskeletal homeostasis (Etienne-Manneville and Hall, Nature 420, 629-635, 2002; Pertz et al., Nature 440, 1069-1072, 2006). To test the hypothesis that AngII-activated TRPC5 channels promote active, GTP-bound Rac1, whereas TRPC6 channels promote active, GTP-bound RhoA, in vitro GTPase activity assays were performed in HEK cells expressing the AT1R and TRPC channels (FIGS. 4A-C). Total lysates from AngII-stimulated cells were affinity isolated with either p21-Activated Kinase (PAK1) or Rhotekin, which are known binding partners of the active, GTP-bound Rac1 or RhoA, respectively. Co-expression of AT1R and TRPC5 in HEK293T cells increased Rac1 activity compared to cells expressing only the AT1R (FIG. 4A, Lane 2 vs. 1, baseline). In contrast, co-expression of AT1R and TRPC6 decreased Rac1 activity and increased RhoA activation relative to baseline (Lane 3 vs. 1). Active RhoA levels were reduced by the co-expression of AT1R and TRPC5 (Lane 2). Taken together, these data suggest a functional dependence of Rac1 on TRPC5 channel activity, and of RhoA on TRPC6 channel activity.

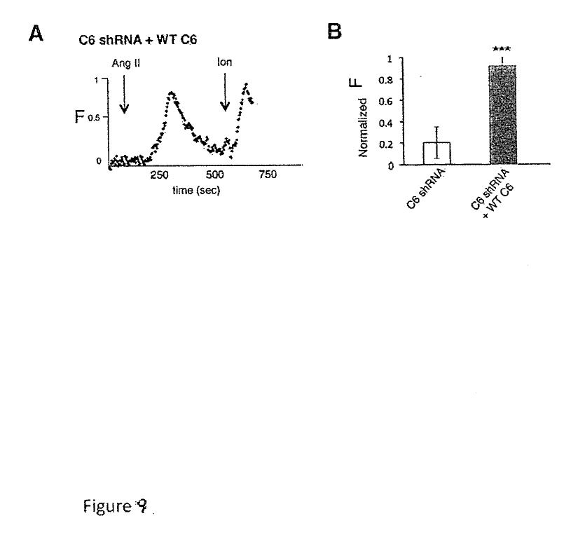

This functional coupling was verified in podocytes. Dominant negative RhoA (DN RhoN19) induces loss of stress fibers, whereas dominant negative Rac1 (DN RacN17) promotes stress fiber formation (Etienne-Manneville and Hall, Nature 420, 629-635, 2002). As expected, in control podocytes (FIG. 4D, a-c), where TRPC5 and TRPC6 channels are intact, DN RhoN19 induced loss of stress fibers and DN RacN17 promoted stress fiber formation. Overexpression of DN RhoN19 in TRPC5-depleted podocytes abolished stress fibers (22±3 vs. 4±1; one way ANOVA Fisher's LSD, P<0.001, n=20) (FIG. 4D, d vs. e; E), thus phenocopying TRPC6 silenced cells (FIG. 4D, g; E). Similarly, treatment of TRPC5 depleted cells with Y27632 (ROCKi, 10 μM), a potent inhibitor of the critical downstream effector RhoA Kinase (ROCK) induced loss of stress fibers (22±3 vs. 6±1) (FIG. 4D, f; E). In contrast, overexpression of DN RacN17 restored prominent stress fibers in TRPC6 silenced cells (21±2 vs. 3±1) (FIG. 4D, g vs. h; E). Expression of wild-type TRPC6 channel on the silenced TRPC6 shRNA background restored a near-maximal Ca²⁺ transient amplitude in Ca²⁺ imaging experiments (FIG. 9) and also resulted in a reversal to a prominent stress fiber network (21±3 vs. 3±1) (FIG. 4B, g vs. I; E). Notably, TRPC5 silenced cells phenocopied control podocytes at baseline, and cells pre-treated with Losartan as compared to Scr shRNA controls (23±2 and 25±1 vs. 16±1) (FIG. 4E).

### Example 5

### Cell Migration Requires TRPC Channel-Mediated Activation of GTPases

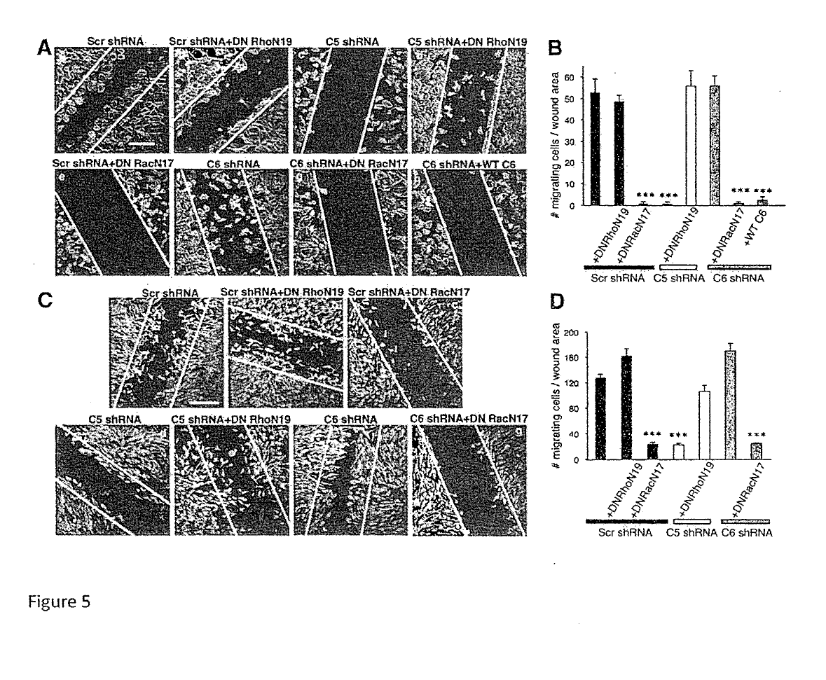

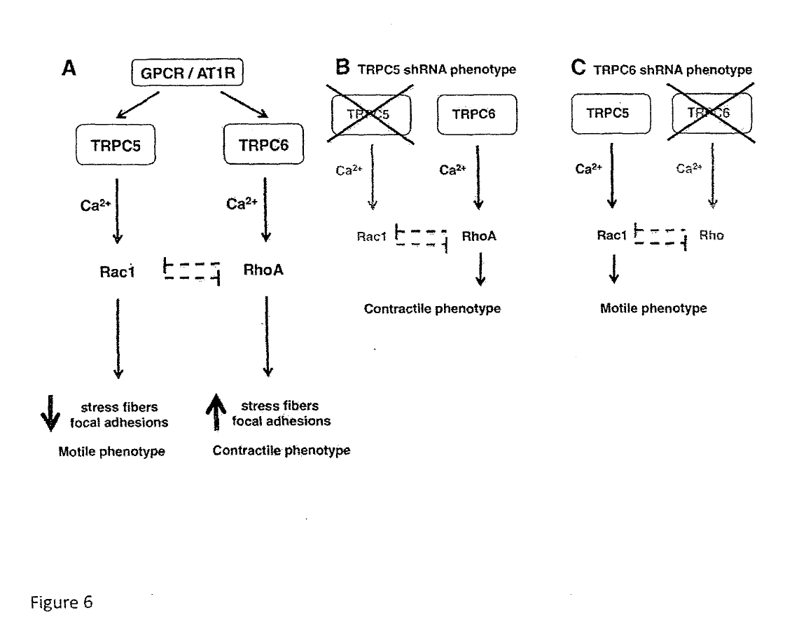

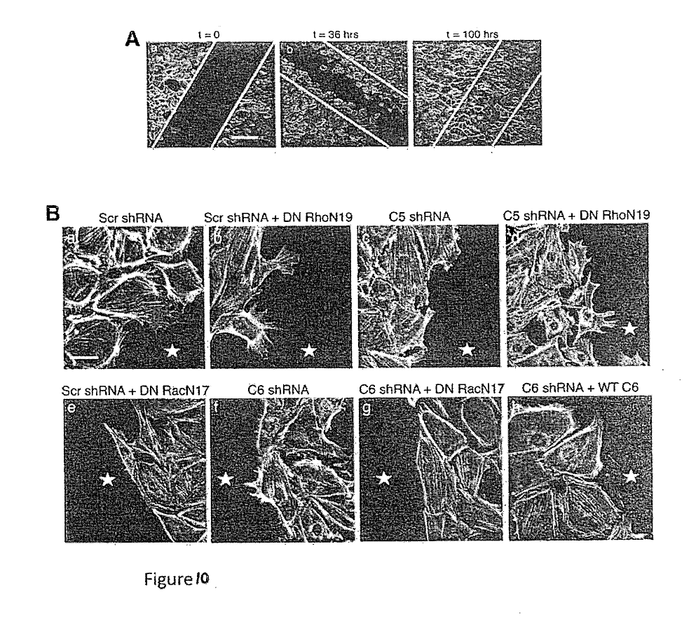

Remodeling of the actin cytoskeleton under the regulation of Rac1 and RhoA coordinates cell motility (Etienne-Manneville and Hall, Nature 420, 629-635, 2002). The contribution of TRPC5 and TRPC6 in podocyte migration was examined using a classic wound assay (Asanuma et al., Nat Cell Biol 8, 485-491, 2006). In control experiments, at 36 hours, podocytes partially closed the wound by migrating as a sheet of cells (FIG. 10), consistent with recent observations (Asanuma et al., Nat Cell Biol 8, 485-491, 2006). At 36 hours, DN RhoN19-induced cell motility was similar to scrambled shRNA controls (52±6 and 48±3; one way ANOVA Fisher's LSD, P<0.001, n=7 images) (FIGS. 5A and 5B). In contrast, DN RacN17 reduced migration into the wound (1±0.3) (FIGS. 5A, a vs. e; 5B). TRPC5 silenced podocytes were unable to migrate into the wound, consistent with a contractile cell phenotype (1±0.4) (FIGS. 5A, c; 5B), whereas TRPC6 silenced cells showed increased motility (56±2) (FIGS. 5A, f; 5B). DN RhoN19 induced previously stationary TRPC5 depleted cells to readily migrate into the wound (55±7) (FIGS. 5A, d; 5B). On the contrary, DN RacN17 impaired TRPC6 depleted podocytes from migrating into the wound (1±0.3) (FIGS. 5A, g; 5B). This reduction in motility was mirrored in cells expressing WT TRPC6 on a background of TRPC6 shRNA (2±1) (FIGS. 5A, h; B).

Finally, to test whether the observed sensor-switch coupling is a general mechanism underlying the regulation of actin dynamics, the role of TRPCs in the regulation of cell migration in fibroblasts was investigated. The expression of TRPC5 and TRPC6 at the mRNA level was confirmed by RT-PCR (FIG. 7C). In migration assays, at 36 hours, DN RhoN19 induced fibroblast motility (162±5) (FIGS. 5C, a vs. b; 5D), whereas DN RacN17 attenuated migration into the wound (24±2; one way ANOVA Fisher's LSD, P<0.001, n=6 images) (FIGS. 5C, a vs. c; 5D). TRPC5 silenced cells were unable to drive wound closure (22±3) (FIGS. 5C, d; 5D). Overexpression of DN RhoN19 in TRPC5 depleted cells induced individual cells to migrate into the wound (116±9) (FIGS. 5C, e; 5D). Consistent with our findings in podocytes, TRPC6 silenced fibroblasts showed significantly increased motility compared to TRPC5 depleted cells (171±11 vs. 22±3), but in contrast to podocytes, they appeared to migrate as a sheet rather than as individual cells (FIGS. 5C, f; 5D). DN RacN17 in TRPC6 silenced fibroblasts impaired their ability to migrate into the wound (25±1) (FIGS. 5C, f vs. g; D). Taken together, these data suggest that the functional connection between TRPC5/Rac1 activity and TRPC6/RhoA activity is a conserved cell biological mechanism for Ca²⁺-mediated regulation of actin remodeling and cell migration.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating, or reducing risk of developing, kidney disease in a subject, the method comprising administering to the subject a therapeutically effective amount of a TRPC5 inhibitor, thereby treating, or reducing risk of developing, kidney disease in the subject.

2. The method of claim 1, wherein the TRPC5 inhibitor is an anti-TRPC5 antibody or antigen-binding fragment thereof.

3. The method of claim 1, wherein the TRPC5 inhibitor is an inhibitory nucleic acid effective to specifically reduce expression of TRPC5.

4. The method of claim 3, wherein the inhibitory nucleic acid is a small interfering RNA molecule or antisense nucleic acid that specifically targets TRPC5.

5. The method of claim 1, wherein the TRPC5 inhibitor is selected from the group consisting of 2-aminoethoxydiphenylborane and 1-oleoyl-2-acetyl-sn-glycerol.

6. The method of claim 1, wherein the subject is a mammal.

7. The method of claim 1, wherein the subject is a human.

8. The method of claim 1, further comprising:
detecting TRPC5 levels in a sample comprising podocytes;
comparing TRPC5 levels in the sample to a reference level of TRPC5; and
administering the TRPC5 inhibitor if the levels of TRPC5 in the sample are elevated as compared to the reference.

9. The method of claim 1, wherein the kidney disease is proteinuria.

10. The method of claim 1, wherein the kidney disease is microalbuminuria or macroalbuminuria.

11. A method of identifying a candidate compound for treating, or reducing risk of developing, kidney disease, the method comprising:
providing a sample comprising a TRPC5 polypeptide;
contacting the sample with a test compound;
determining a level of calcium ion (Ca²⁺) transport in the sample in the presence of the test compound; and
if the test compound decreases the level of Ca²⁺ transport, relative to a level of Ca²⁺ transport in the absence of the test compound, then the test compound is a candidate compound for treating, or reducing risk of developing, kidney disease.

12. The method of claim 11, wherein the kidney disease is proteinuria.

13. The method of claim 11, wherein the kidney disease is microalbuminuria or macroalbuminuria.

14. The method of claim 10, wherein the sample is a living cell.

15. The method of claim 10, further comprising:
selecting a candidate compound;
administering the candidate compound to a mammal; and
evaluating an effect of the candidate compound on kidney disease,
wherein a candidate compound for treating, or reducing risk of developing, kidney disease, is a candidate therapeutic agent for the treatment of kidney disease.

16. The method of claim 15, wherein the kidney disease is proteinuria.

17. The method of claim 15, wherein the kidney disease is microalbuminuria or macroalbuminuria.


SEQUENCE LISTING (6 sequences)

SEQ ID NO: 1 - DNA, 58 bp, Artificial Sequence
  Synthesized in the laboratory
ccggcctggcaactatttccctgaactcgagttcagggaaatagttgccaggtttttg

SEQ ID NO: 2 - DNA, 58 bp, Artificial Sequence
  Synthesized in the laboratory
ccgggcaatcaaataccaccagaaactcgagtttctggtggtatttgattgctttttg

SEQ ID NO: 3 - DNA, 58 bp, Artificial Sequence
  Synthesized in the laboratory
ccggcccaagtcatttctataccttctcgagaaggtatagaaatgacttgggtttttg

SEQ ID NO: 4 - DNA, 58 bp, Artificial Sequence
  Synthesized in the laboratory
ccgggctcattatatcctgggtaatctcgagattacccaggatataatgagctttttg

SEQ ID NO: 5 - DNA, 58 bp, Artificial Sequence
  Synthesized in the laboratory
ccggcgttctgtatggtgtctataactcgagttatagacaccatacagaacgtttttg

SEQ ID NO: 6 - DNA, 58 bp, Artificial Sequence
  Synthesized in the laboratory
ccggcgtccaaatctcagccgtttactcgagtaaacggctgagatttggacgtttttg